(19) United States
(12) Patent Application Publication
(10) Pub. No.: US 2015/0306357 A1
(43) Pub. Date: Oct. 29, 2015

(54) GUIDEWIRE

(71) Applicant: ASAHI INTECC CO., LTD., Nagoya-shi (JP)

(72) Inventors: Satoru MURATA, Seto-shi (JP); Tadahiro KOIKE, Ama-shi (JP); Keisuke USHIDA, Nagoya-shi (JP)

(21) Appl. No.: 14/695,838

(22) Filed: Apr. 24, 2015

(30) Foreign Application Priority Data
Apr. 24, 2014 (JP) 2014-089715

(51) Int. Cl. A61M 25/09

(52) U.S. Cl. CPC A61M 25/09; A61M 2025/09191

(57) ABSTRACT

A guidewire includes a shaft, an outer coil wound around a distal end portion of the shaft, and an inner coil provided within the outer coil. The outer coil is formed by winding a plurality of stranded wires in a spiral manner, each of the stranded wires being formed of a plurality of elemental wires twisted together, and the direction of winding of the outer coil is opposite to the direction of winding of the inner coil.

### CROSS REFERENCE TO RELATED APPLICATION

This application claims priority to Japanese Patent Application No. 2014-089715 filed in the Japan Patent Office on Apr. 24, 2014, the entire contents of which are incorporated herein by reference.

### BACKGROUND

The disclosed embodiments relate to a guidewire for use as medical equipment that is inserted into body cavities for the purpose of treatment and examination.

Guidewires are known in the art for use as a guide for a catheter, or the like, that is inserted into tubular organs, such as blood vessels, the digestive tract, and the ureter, and into body tissue for the purpose of treatment and examination. For example, Japanese Patent Application Publication No. 8-317989 (JP 8-317989 A) discloses a traditional guidewire that includes a core wire, an outer coil provided at a distal end portion of the core wire, and an inner coil provided within the outer coil.

### SUMMARY

When torque is applied to the guidewire of Japanese Patent Application Publication No. 8-317989 (JP 8-317989 A) in such a direction that the outer coil becomes tightened, elemental wires of the outer coil are pressed against each other. This causes contact pressure of the outer coil to increase so that the outer coil deforms inwardly to reduce its diameter. When such deformation occurs to an excessive degree, the elemental wires are shifted and become displaced onto adjacent elemental wires. Thus, the guidewire of JP 8-317989 A does not have sufficient torque transmission to be inserted deep into a firm lesion of a patient without deforming.

Embodiments of the present disclosure address these deficiencies of the traditional guidewires. In the embodiments, a guidewire coil is formed by winding a plurality of stranded wires, each formed of a plurality of elemental wires twisted together, in a spiral manner.

A guidewire of the disclosed embodiments includes a shaft, an outer coil wound around a distal end portion of the shaft, and an inner coil provided within the outer coil. The outer coil is formed of a plurality of stranded wires wound in a spiral manner, each of the stranded wires being formed of a plurality of elemental wires twisted together. A winding direction of the outer coil is opposite to a winding direction of the inner coil.

Usually, when torque is applied to a guidewire in such a direction that the outer coil becomes tightened, the elemental wires are pressed together and the stranded wires are pressed together. This increases contact pressure of the outer coil, which causes the outer coil to deform inwardly to reduce its diameter. When such deformation occurs to an excessive degree, an elemental wire (stranded wire) becomes shifted and displaced onto an adjacent elemental wire (stranded wire). Such shifting and displacement reduces operability of the outer coil, and thus of the guidewire.

In the disclosed embodiments, the winding direction of the outer coil is opposite to the winding direction of the inner coil and therefore, when winding of the outer coil is tightened and the outer coil deforms inwardly to reduce its diameter, the inner coil is relaxed. Thus, the elemental wires of the inner wire are in close contact so that the outer diameter of the inner coil increases. This leads both of the coils to interfere with each other to suppress excessive inward deformation of the outer coil. As a result, such a problem described above that an elemental wire (stranded wire) becomes shifted and displaced onto an adjacent elemental wire (stranded wire) can be avoided.

### BRIEF DESCRIPTION OF THE DRAWINGS

FIG. 1 schematically illustrates a partial cross-section of a guidewire according to embodiments;

FIG. 2 is a sectional view taken from line A-A of FIG. 1;

FIG. 3 is a cutaway side view of the guidewire of FIG. 1;

FIG. 4 schematically illustrates a partial cross-section of a guidewire according to embodiments;

FIG. 5 is a perspective view of an inner coil of the guidewire of FIG. 4;

FIG. 6 is a sectional view taken from line B-B of FIG. 4;

FIG. 7 schematically illustrates a partial cross-section of a guidewire according to embodiments; and

FIG. 8 is a sectional view taken from line C-C of FIG. 7.

### DETAILED DESCRIPTION OF EMBODIMENTS

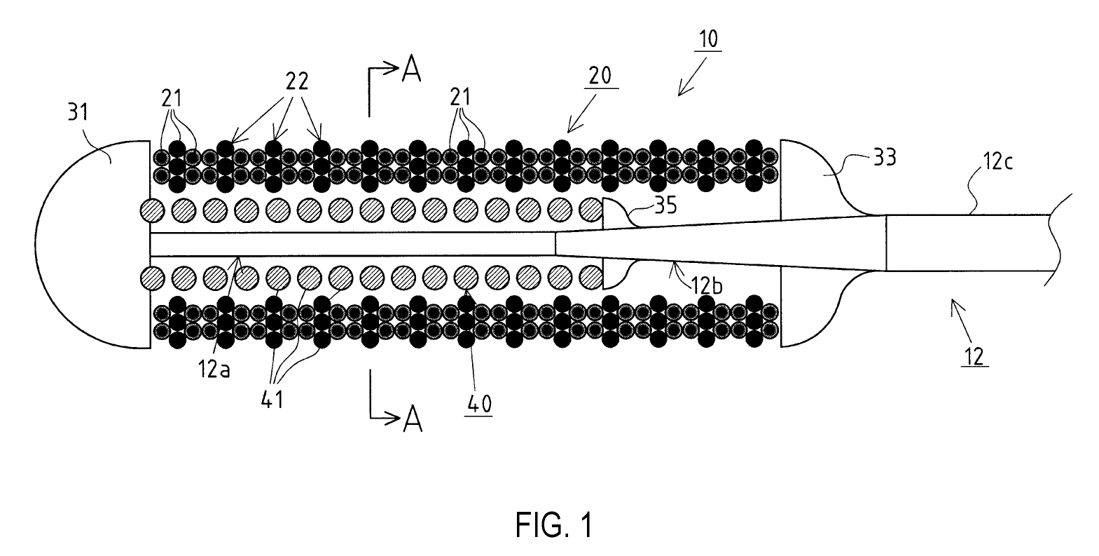

FIG. 1 is an expanded view of a partial cross-section of a guidewire 10 according to embodiments. In FIG. 1, the distal end side, which is to be inserted into a patient's body, is shown on the left hand side, and the proximal end side, which is to be handled by an operator such as a doctor, is shown on the right hand side. The drawings are merely representations of the disclosed embodiments, and the relative sizes of the components is not limited to those depicted in the drawings.

The guidewire 10 may be used, for example, for treating blood vessels of a lower limb with the Cross Over technique. As shown in FIG. 1, the guidewire 10 includes a shaft 12 and an outer coil 20 covering an outer circumference of a distal end portion of the shaft 12.

The shaft 12 includes a thin portion 12, a tapered portion 12b, and a greater-diameter portion 12c. The thin portion 12a is distal of the tapered portion 12b, and the tapered portion 12b is distal of the greater-diameter portion 12c. The thin portion 12a may be located at the most distal end side of the shaft 12 and may be the most flexible part of the shaft 12. The thin portion 12a may be formed flat by pressing, as is known by one of skill in the art. The tapered portion 12b may be tapered with a circular cross section such that its diameter is reduced toward the distal end side of the shaft 12. The greater-diameter portion 12c may have a diameter greater than the diameter of the thin portion 12a.

The material of the shaft 12 is not particularly limited and may include, for example, a stainless steel (SUS304), a super-elastic alloy such as Ni—Ti alloys, piano wire, a cobalt-based alloy, or a mixture of these materials.

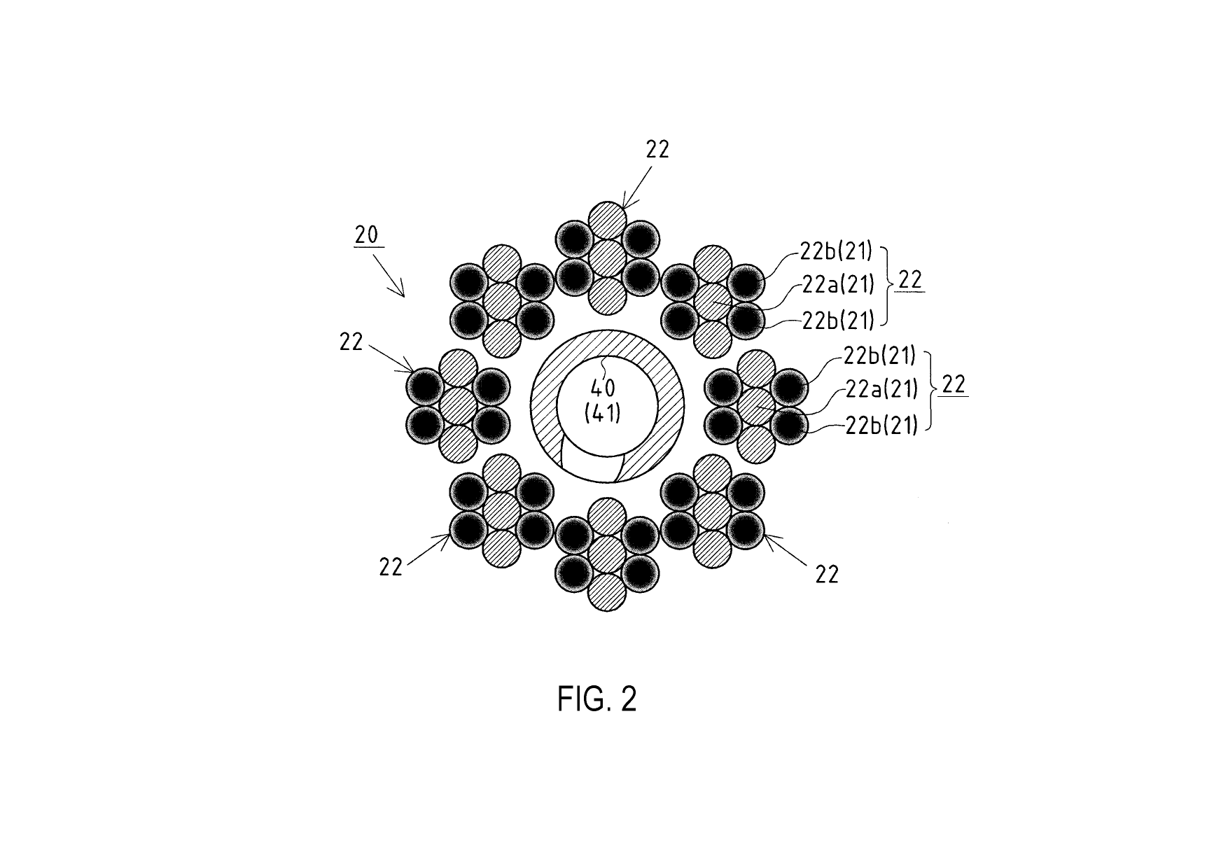

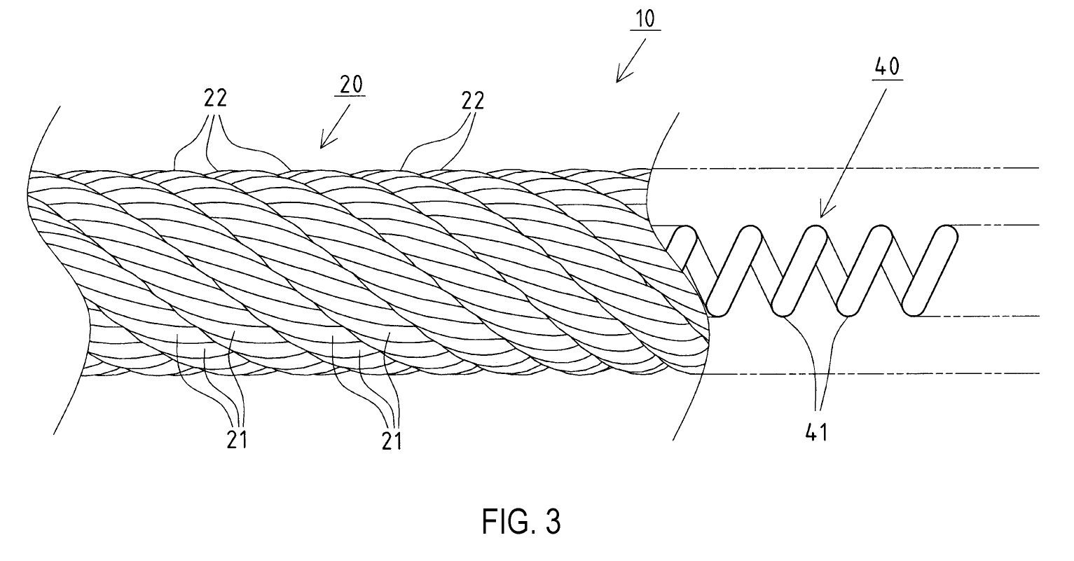

As shown in FIGS. 1 and 2, the outer coil 20 may be formed by winding a plurality of stranded wires 22 in a spiral manner In FIG. 2, 8 stranded wires 22 are shown. However, various numbers of stranded wires 22 may be used, including, for example, 2, 4, 7, or 11 stranded wires 22. As shown in FIG. 2, each of the stranded wires 22 includes a core wire 22a and peripheral wires 22b covering the outer circumference of the core wire 22a. In FIG. 2, 6 peripheral wires 22b are shown. However, various numbers of peripheral wires 22b may be used. As shown in FIG. 3, the winding direction of the outer coil 20 may be clockwise (i.e., to the right in FIG. 2).

The material of the core wire 22a and of the peripheral wires 22b is not particularly limited and may include, for example, stainless steels such as martensitic stainless steel, ferritic stainless steel, austenitic stainless steel, austenitic-ferritic duplex stainless steel, and precipitation hardened stainless steel, super-elastic alloys such as Ni—Ti alloys, and metals radiopaque to X-rays such as platinum, gold, tungsten, tantalum, and iridium, and alloys thereof. Additionally, the material of the core wire 22a and of the peripheral wires 22b may be a mixture of two or more materials. The core wire 22a may be formed of the same or of different material(s) from the peripheral wires 22b. One or more peripheral wires 22b may be formed of different material(s) from another peripheral wire 22b.

The distal end of the outer coil 20 may be fixed to the distal end of the shaft 12 via a distal end bonding member 31, as shown in FIG. 1. The proximal end of the outer coil 20 may be fixed to the shaft 12 via a proximal end bonding member 33. The material of the distal end bonding member 31 and of the proximal end bonding member 33 is not particularly limited and may include, for example, brazing metals such as Sn—Pb alloys, Pb—Ag alloys, Sn—Ag alloys, and Au—Sn alloys. The material of the distal end bonding member 31 and of the proximal end bonding member 33 may be a mixture of two or more materials. The distal end bonding member 31 may be formed of the same or of different material(s) from the proximal end bonding member 33.

The guidewire 10 may also include an inner coil 40 within the outer coil 20. The inner coil 40 may be a single-strand coil formed by winding an elemental wire 41 in a spiral manner.

The material of the inner coil 40 is not particularly limited and may include, for example, a radiopaque elemental wire or a radiolucent elemental wire. The material of the radiopaque elemental wire may include gold, platinum, tungsten, an alloy containing such an element (a platinum-nickel alloy, for example), or the like. The material of the radiolucent elemental wire may include stainless steel (SUS304 or SUS316, for example), a super-elastic alloy such as Ni—Ti alloys, piano wire, or the like. The material of the radiopaque elemental wire and/or of the radiolucent elemental wire may be a mixture of two or more materials.

The distal end of the inner coil 40 may be bonded to the distal end of the shaft 12 via the distal end bonding member 31. The proximal end of the inner coil 40 may be bonded to the shaft 12 via a proximal end bonding member 35. The material of the proximal end bonding member 35 is not particularly limited and may include, for example, brazing metals such as Sn—Pb alloys, Pb—Ag alloys, Sn—Ag alloys, and Au—Sn alloys. The proximal end bonding member 35 may be formed of the same or of different materials from the distal end bonding member 31 and/or from the proximal end bonding member 33.

As shown in FIG. 3, the winding direction of the inner coil 40 may be counterclockwise (i.e., to the right in FIG. 3). In other words, the winding direction of the outer coil 20 may be opposite to the winding direction of the inner coil 40.

Upon the application of torque to traditional guidewires, an outer coil may become tightened. Such tightening may cause wires of the outer coil to be pressed together. This may increase contact pressure between the wires of the outer coil, which may consequently cause the outer coil to deform inwardly to reduce the diameter of the outer coil. When such deformation occurs to an excessive degree, the wires may shift position and become displaced.

In the disclosed embodiments, the winding direction of the outer coil 20 is opposite to the winding direction of the inner coil 40 so that the wires of the guidewire 10 resist shifting of their position and resist being displaced when torque is applied to the guidewire 10. For example, due to the application of torque to the guidewire 10, the winding of the outer coil 20 may be tightened and the outer coil 20 may slightly deform inwardly to reduce its diameter. However, the inner coil 40 remains relaxed and does not deform inwardly. Thus, the inner coil 40 may be in close contact with the slightly deformed outer coil 20 so that the inner coil 40 prevents the outer coil 20 from excessive inward deformation of the outer coil 20. As a result, the wires of the guidewire (i.e., the elemental wires 20) do not shift position and do not become displaced.

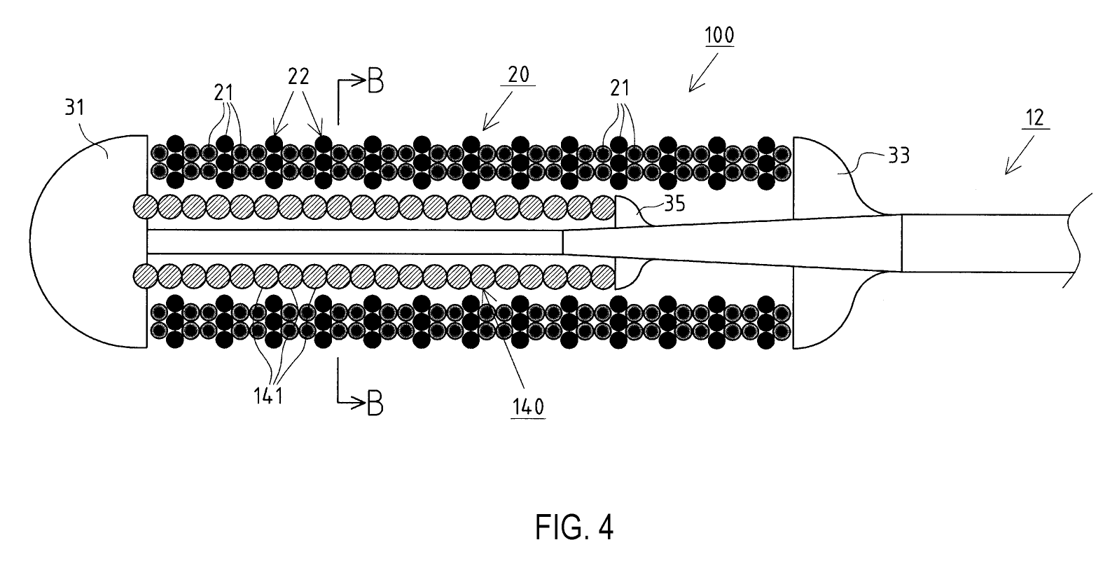

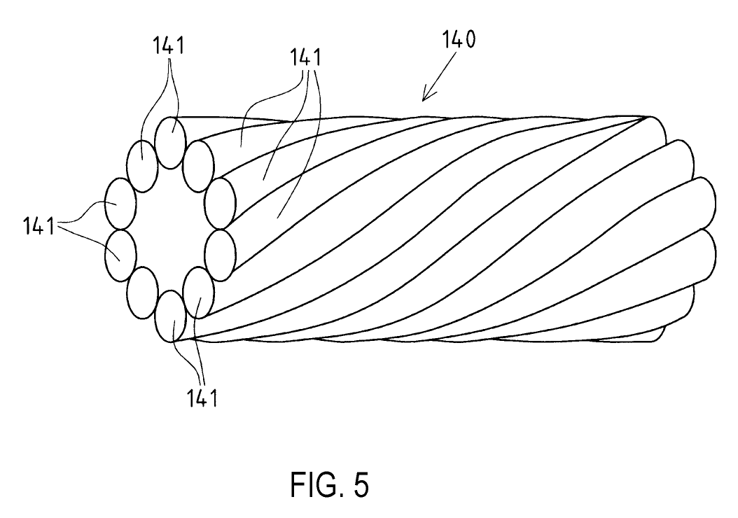

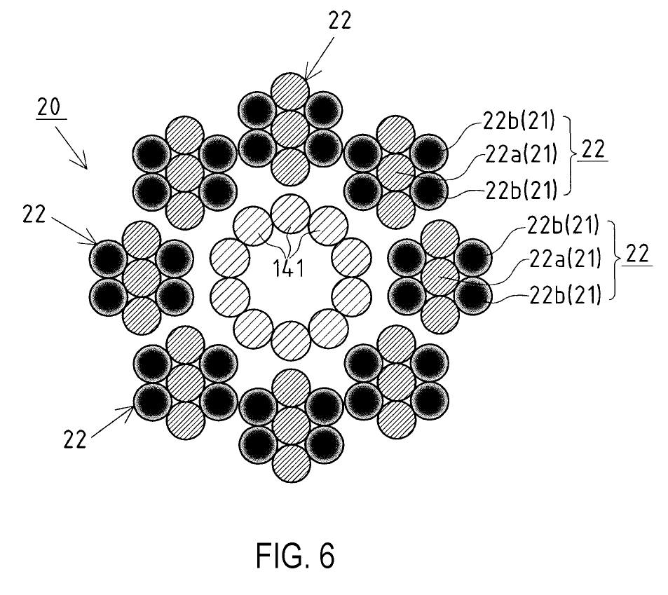

As shown in FIGS. 4, 5 and 6, guidewire 100 may include an inner coil 140 that comprises a single-strand coil formed by winding/twisting elemental wires 141 in a spiral manner The inner coil 140 may be hollow. As shown in FIGS. 5 and 6, 10 elemental wires 141 may be used. However, various numbers of elemental wires 141 may be used, including for example, 2, 4, 7, or 11 elemental wires 141.

The elemental wires 141 may be capable of slightly moving relative to each other. Thus, when torque is applied to the guidewire 100 so that the outer coil 20 is tightened and deforms slightly inward to reduce its diameter, the elemental wires 141 may remain relaxed. Thus, the diameter of the inner coil 140 may increase so that the inner coil 140 may contact the outer coil 20 and may prevent excessive inward deformation of the outer coil 20. Therefore, the elemental wires 21 may not shift position or become displaced.

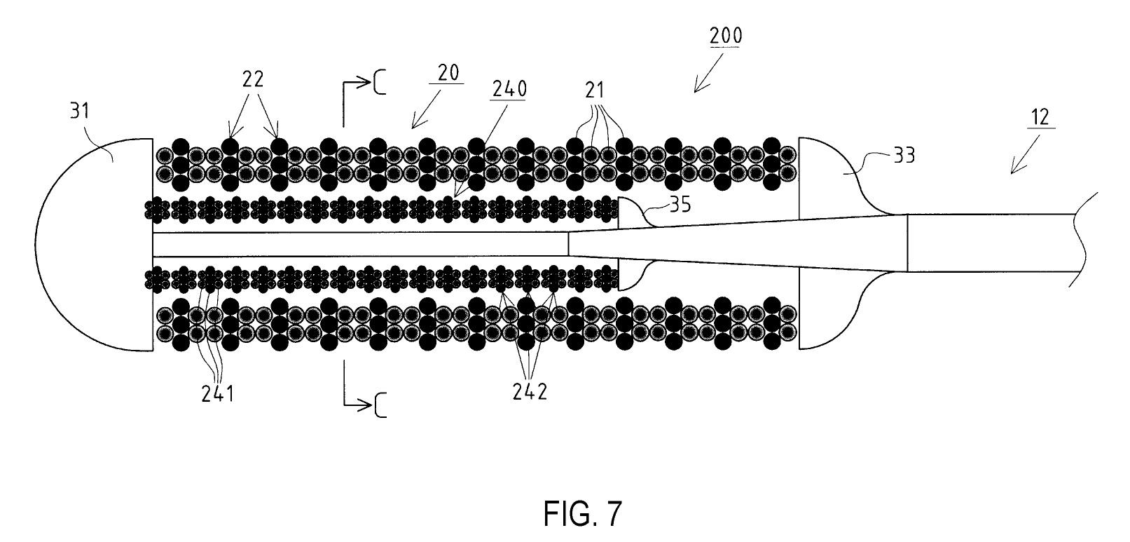

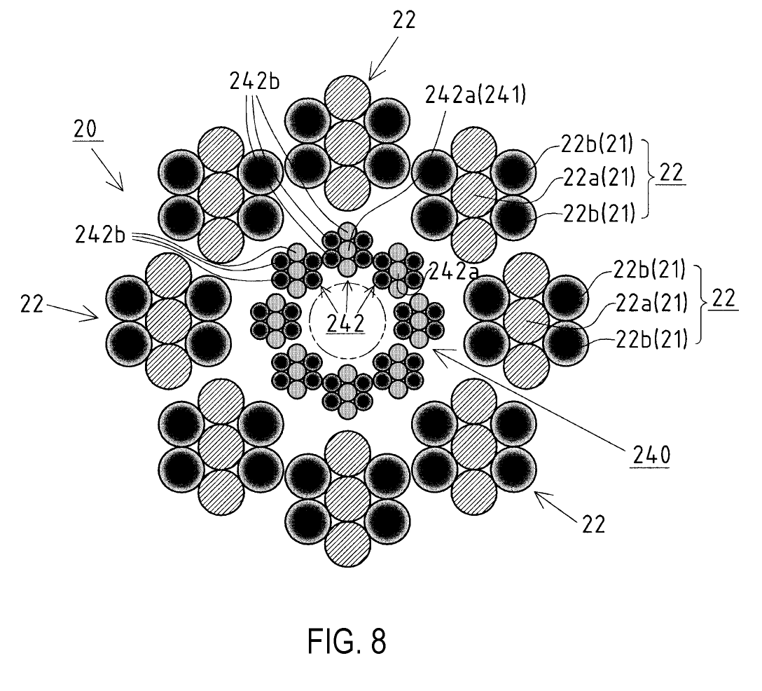

As shown in FIGS. 7 and 8, guidewire 200 may include an inner coil 240 that comprises a plurality of stranded wires 242 wound/twisted a spiral manner. In FIG. 8, 8 stranded wires 242 are shown. However, various numbers of stranded wires 242 may be used, including, for example, 2, 4, 7, or 11 stranded wires 242. The stranded wires 242 may each be formed of a core wire 242a wound together with peripheral wires 242b. As shown in FIG. 8, the peripheral wires 242b may cover the outer circumference of the core wire 242a in a spiral manner. In FIG. 8, 6 peripheral wires 242b are shown. However, various numbers of peripheral wires 242b may be used.

The material of the core wire 242a and of the peripheral wires 242b in the inner coil 240 is not particularly limited and may include, for example, stainless steels such as martensitic stainless steel, ferritic stainless steel, austenitic stainless steel, austenitic-ferritic duplex stainless steel, and precipitation hardened stainless steel, super-elastic alloys such as Ni—Ti alloys, and metals radiopaque to X-rays such as platinum, gold, tungsten, tantalum, and iridium and alloys thereof The material of the core wire 242a and of the peripheral wires 242b may be a mixture of two or more materials. The core wire 242a may be formed of the same or of different material(s) from the peripheral wires 242b. One or more peripheral wires 242b may be formed of different material(s) from another peripheral wire 242b.

The stranded wires 242, and also the elemental wires 241 that form the stranded wires 242, are capable of slightly moving relative to each other. Therefore, the inner coil 240 has a degree of freedom so that the inner coil 240 has improved flexibility. When torque is applied to the guidewire 200, the inner coil 240 remains relaxed so that its outer diameter may increase. The improved flexibility of the inner coil 240 allows the outer diameter of the inner coil 240 to increase to a wide range of expansion. Thus, when winding of the outer coil 20 is tightened and the outer coil 20 deforms inwardly so that its diameter is reduced, the outer diameter of the inner coil 240 may significantly increase. This results in the inner coil 240 contacting and interfering with the outer coil 20 to prevent excessive inward deformation of the outer coil 20. Thus, elemental wires 21 do not shift position or become displaced.

What is claimed is:

1. A guidewire comprising:
a shaft,
an outer coil wound around a distal end portion of the shaft, and
an inner coil provided within the outer coil,
wherein the outer coil is formed of a plurality of stranded wires wound in a spiral manner, each of the stranded wires being formed of a plurality of elemental wires twisted together, and
a winding direction of the outer coil is opposite to a winding direction of the inner coil.

2. The guidewire according to claim 1, wherein
the inner coil is formed of a stranded wire formed of a plurality of elemental wires twisted together.

3. The guidewire according to claim 1, wherein
the inner coil is formed of a plurality of stranded wires wound in a spiral manner, and each stranded wire is formed of a plurality of elemental wires twisted together.

4. The guidewire according to claim 1, further comprising a distal end bonding member that fixes a distal end of the outer coil to a distal end of the shaft.

5. The guidewire according to claim 4, wherein the distal end bonding member bonds a distal end of the inner coil to the distal end of the shaft.

6. The guidewire according to claim 1, further comprising a first proximal end bonding member that fixes a proximal end of the outer coil to the shaft.

7. The guidewire according to claim 6, further comprising a second proximal end bonding member that bonds a proximal end of the inner coil to the shaft.

8. The guidewire according to claim 7, wherein the second proximal end bonding member is distal of the first proximal end bonding member.

9. The guidewire according to claim 1, wherein the shaft includes a smaller-diameter portion, a tapered portion that is proximal of the smaller-diameter portion, and a greater-diameter portion that is proximal of the tapered portion.

10. A guidewire comprising:
a shaft,
an outer coil wound around a distal end portion of the shaft,
an inner coil provided within the outer coil, the inner coil being formed of a plurality of first stranded wires wound in a spiral manner such that each first stranded wire is formed of a plurality of first elemental wires twisted together, and
a distal end bonding member that fixes a distal end of the outer coil to a distal end of the shaft and that bonds a distal end of the inner coil to the distal end of the shaft,
wherein the outer coil is formed of a plurality of second stranded wires wound in a spiral manner, each of the second stranded wires being formed of a plurality of second elemental wires twisted together, and
a winding direction of the outer coil is opposite to a winding direction of the inner coil.
